(19) United States
(12) Patent Application Publication
(10) Pub. No.: US 2024/0191610 A1
(43) Pub. Date: Jun. 13, 2024

(54) METHOD AND SYSTEM FOR DETERMINING EQUIVALENT CIRCULATING DENSITY OF A DRILLING FLUID USING IMAGE-BASED MACHINE LEARNING

(71) Applicant: SAUDI ARABIAN OIL COMPANY, Dhahran (SA)

(72) Inventors: Mohammed K. Albassam, Alkhobar (SA); Arturo Magana-mora, Dhahran (SA); Chinthaka Pasan Gooneratne, Dhahran (SA)

(73) Assignee: SAUDI ARABIAN OIL COMPANY, Dhahran (SA)

(21) Appl. No.: 18/078,568

(22) Filed: Dec. 9, 2022

(51) Int. Cl. E21B 44/00; E21B 21/06; E21B 49/00; G01N 33/28; G06T 7/00; G06T 7/62

(52) U.S. Cl. CPC E21B 44/00; E21B 21/065; E21B 49/005; G01N 33/2823; G06T 7/0004; G06T 7/62; E21B 2200/20; E21B 2200/22; G06T 2207/20081; G06T 2207/20084; G06T 2207/30108

(57) ABSTRACT

A method may include obtaining a cutting image data using an image capturing device. The cutting image may be obtained from drilling fluid cuttings circulated out of a wellbore. The method may include obtaining drilling operation data in real-time regarding the drilling system and obtaining drilling fluid data. The method may include determining a cutting parameter of the cuttings by using the cutting image data and an image processing function. The method may include determining a lithology parameter of the cuttings using the cutting image data and a machine-learning model. The method may include determining an equivalent circulating density (ECD) value of the drilling fluid using an ECD model, the cutting parameter, the lithology parameter, the drilling operation data, and the drilling fluid data. The method may include transmitting a command that adjusts a drilling parameter based on the ECD value of the drilling fluid.

### BACKGROUND

Drilling fluid, also called drilling mud, may be a heavy, viscous fluid mixture that is used in oil and gas drilling operations to carry rock cuttings from a wellbore back to the surface. Drilling mud may also be used to lubricate and cool a drill bit. The drilling fluid, by hydrostatic pressure, may also assist in preventing the collapse of unstable strata into the wellbore as well as the intrusion of water from stratigraphic formations proximate the wellbore.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method that includes obtaining, using an image capturing device, cutting image data regarding various cuttings carried by a drilling fluid circulating in a wellbore during a drilling operation. The method further includes obtaining drilling operation data in real-time regarding a drilling system performing the drilling operation. The method further includes obtaining drilling fluid data regarding the drilling fluid. The method further includes determining, by a computer processor, a cutting parameter of the cuttings using the cutting image data and an image processing function. The method further includes determining, using the computer processor, a lithology parameter of the cuttings using the cutting image data and a machine-learning model. The method further includes determining, by the computer processor, an equivalent circulating density (ECD) value of the drilling fluid using an ECD model, the cutting parameter, the lithology parameter, the drilling operation data, and the drilling fluid data. The method further includes transmitting, by the computer processor, a command that adjusts a drilling parameter of the drilling operation based on the ECD value of the drilling fluid.

In general, in one aspect, embodiments relate to a system that includes an image capturing device disposed on a drilling fluid circulation path of a wellbore during a drilling operation. The system further includes a drilling system including a drill string and various sensors coupled to the wellbore. The system further includes a mud pump system coupled to the wellbore. The mud pump system supplies drilling fluid to the wellbore. The system further includes a server coupled to the image capturing device, the drilling system, and the mud pump system. The server includes a computer processor. The server obtains cutting image data regarding various cuttings carried by drilling fluid circulating in the well using the image capturing device. The server also obtains drilling operation data in real-time regarding a drilling system performing the drilling operation. The server obtains drilling fluid data regarding the drilling fluid. The server further determines a cutting parameter of the cuttings using the cutting image data and an image processing function. The server further determines a lithology parameter of the cuttings using the cutting image data and a machine-learning model. The server further determines an equivalent circulating density (ECD) value of the drilling fluid using an ECD model, the cutting parameter, the lithology parameter, the drilling operation data, and the drilling fluid data.

In some embodiments, the image capturing device is a camera device coupled to a shale shaker. In some embodiments, the cutting parameter describes a cuttings geometry of a portion of various cuttings. In some embodiments, the cutting parameter corresponds to a volume concentration of various cuttings. In some embodiments, the machine-learning model is a region-based convolutional neural network (R-CNN). In some embodiments, the ECD value is determined by an edge server disposed at a well site including a wellbore. In some embodiments, drilling operation data includes a rate of penetration of a drill string in a drilling operation. In some embodiments, the drilling fluid data includes rheological data regarding the drilling fluid. In some embodiments, a user device is coupled to the server and the user device provides a graphical user interface for presenting the ECD value to a user. The user device obtains one or more user selections regarding an adjusted rate of penetration value in response to presenting the ECD value. In some embodiments a shale shaker is coupled to the image capturing device and the image capturing device is a camera device. In some embodiments the cutting parameter describes a cuttings geometry of a portion of the cuttings and the cutting parameter corresponds to a volume concentration of the cuttings. In some embodiments the machine-learning model is a region-based convolutional neural network (R-CNN).

In some embodiments a control system is coupled to the server and the server adjusts a drilling parameter or a drilling fluid parameter based on the ECD value. The server also obtains training data including various labeled images of cuttings, and a respective labeled image among the labeled images corresponds to a predetermined lithology. The server also performs a training operation of the machine-learning model using the training data. In some embodiments, the server determines cutting image data regarding the cuttings of the drilling fluid, determines cutting geometry data of the cuttings based on the cutting image data, and determines a slip rate of the cuttings using the cutting geometry data. In some embodiments, the system determines the ECD value based on the slip rate of the second set of cuttings. In some embodiments, the drilling operation data is a rate of penetration of a drill string in the drilling operation.

In light of the structure and functions described above, embodiments disclosed herein may include respective means adapted to carry out various steps and functions defined above in accordance with one or more aspects and any one of the embodiments of one or more aspect described herein.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIGS. 1, 2, and 3 show systems in accordance with one or more embodiments.

FIG. 4 shows a flowchart in accordance with one or more embodiments.

FIGS. 5 and 6 show examples in accordance with one or more embodiments.

FIG. 7 shows a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure include systems and methods for processing cutting image data from one or more image capturing devices using image processing functions and/or machine-learning models. For example, an image capturing device may be an Internet of Things (IOT) network device that includes one or more cameras for acquiring cutting image data based on cuttings in drilling fluid, such as cuttings acquired by a shale shaker. The image capturing device may include one or more communication interfaces for transmitting cutting image data to a server or other location in a well network for further data processing. In particular, the cutting image data may be used to determine one or more lithological parameters (e.g., lithological characteristics of a formation that produced the cuttings) and/or one or more cutting parameters (e.g., the size, shape, and volume concentration of the cuttings as well as cutting slip velocity).

In some embodiments, cutting image data is used to determine one or more equivalent circulating density (ECD) values of drilling fluid being circulating in a wellbore in a drilling operation. ECD values may describe the effective density of a drilling fluid that results from the sum of hydrostatic pressures imposed by a static fluid column and friction pressure. Thus, a camera-based cuttings analysis tool may be used to accurately determine cutting information from cutting image data which can provide various data inputs to the ECD model. For illustration, cutting slip velocity may affects the time required to circulate cuttings out of a wellbore. Low slip velocities may result in an easier removal of cuttings while also reducing the effect of cuttings on ECD. However, if cuttings slip velocity is relatively high, then more time may be required to remove cuttings from the well.

In some embodiments, cutting image data, drilling operation data, and/or drilling fluid data are collected at a drilling rig site using an edge server. For example, an edge server may use edge computing techniques on location at a drilling site where cutting image data, drilling operation data, and/or drilling fluid data are acquired at the edge of a well network. Rather than sending cutting image data, drilling operation data, and/or drilling fluid to a remote datacenter or cloud server for data processing, the edge server may perform data processing locally at the drilling site, such as to determine cutting parameters and/or lithological parameters. As such, IoT devices and edge computing may be used to rapidly analyze data in real-time during a drilling operation, such as by an automated drilling manager. By processing cutting image data and other acquired data on a well network, the well network may use this processed data to adjust drilling parameters and/or drilling fluid parameters accordingly.

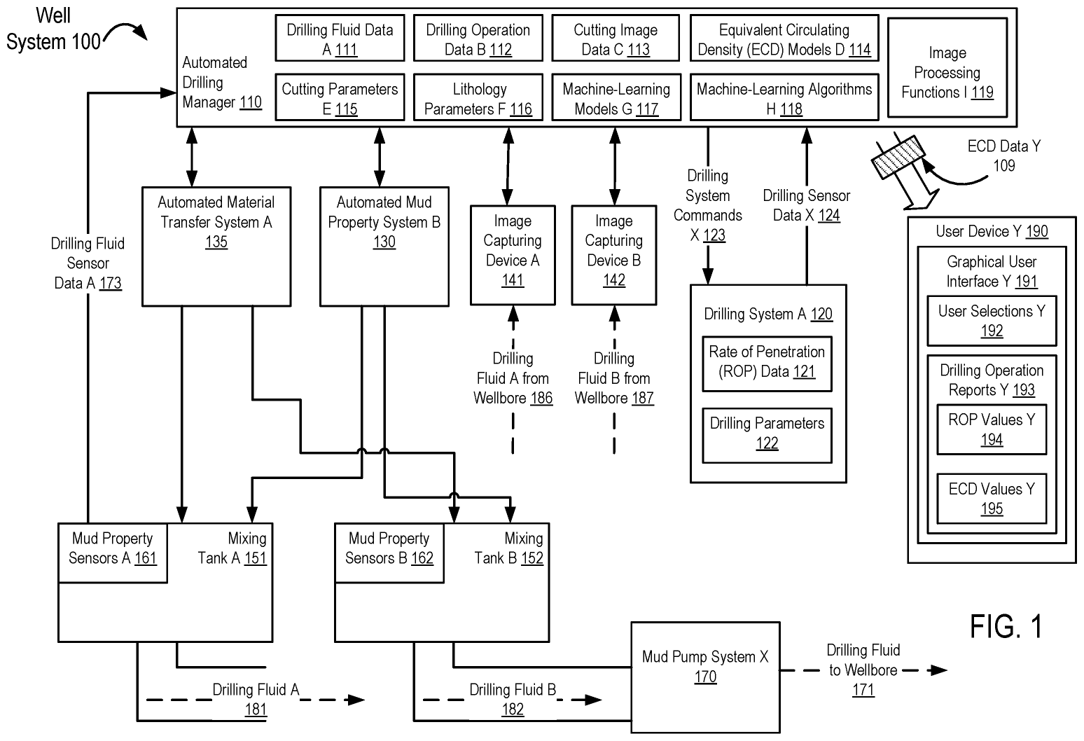

Turning to FIG. 1, FIG. 1 shows a schematic diagram in accordance with one or more embodiments. As shown in FIG. 1, FIG. 1 illustrates a well system (100) that may include an automated drilling manager (e.g., automated drilling manager (110)) coupled to one or more user devices (e.g., user device Y (190)), a drilling system (e.g., drilling system A (120)), a mud pump system (e.g., mud pump system X (170)), an automated material transfer system (e.g., automated material transfer system A (135)), an automated mud property system (e.g., automated mud property system B (130)), and various drilling fluid processing components. For example, drilling fluid processing equipment may include one or more feeders, one or more control valves, one or more mixing tanks (e.g., mixing tank A (151), mixing tank B (152)), and a solid removal system that may include one or more shale shakers. An automated mud property system may include hardware and/or software that includes functionality for monitoring and/or controlling various chemical components used to produce drilling fluid. Likewise, the automated drilling manager may include hardware and/or software for monitoring and/or controlling one or more drilling operations performed by a drilling system. For example, the automated drilling manager may be an edge server or fog server that provides an entry point into a well network. An edge server may be a type of network device that provides an entry point into a well network, where other edges devices in the well network may include various routers and network switches.

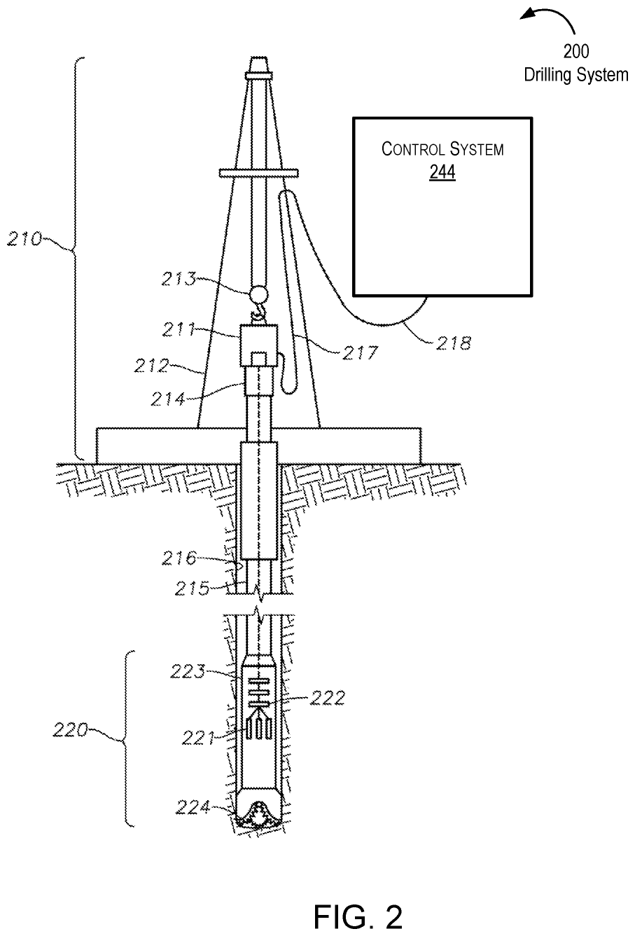

With respect to the drilling system, drilling fluid may circulate through a drill string for continuous drilling, e.g., drilling fluid A (181) and drilling fluid B (182) as shown in FIG. 1, in order to circulate through a wellbore (e.g., drilling fluid to wellbore (171)). In particular, the ability of the drilling fluid to carry drilled cuttings from a wellbore may be governed by several factors that relate to various drilling fluid properties (e.g., mud rheology, mud weight, etc.) and various drilling operation parameters (e.g., drilling parameters (122)) such as drill pipe rotary speed (RPM), pipe eccentricity (i.e., axial location of the drill pipe), hole inclination angle, rate of penetration (ROP) (e.g., with respect to ROP data (121)). Likewise, used drilling fluid from a wellbore may be passed through a solid removal system prior to entering a mixing tank or being sent to a mud pump system. More specifically, a solid removal system may include equipment and other hardware for removing particular solids, such as drill cuttings and coarse aggregates, from used drilling fluid in order to recycle drilling fluid. For more information on drilling systems, see FIG. 2 and the accompanying description below.

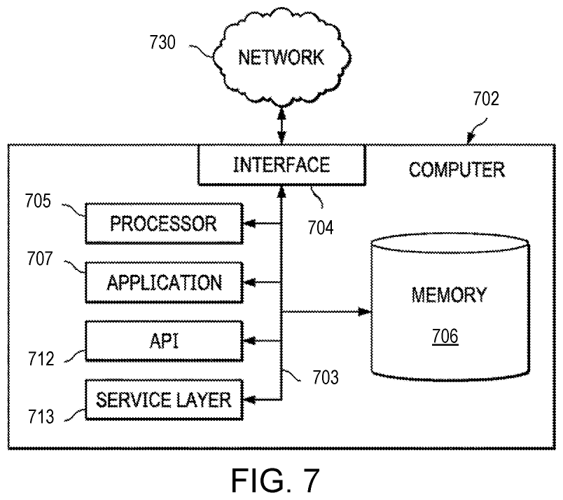

In some embodiments, cutting image data of various cuttings (e.g., cutting image data C (113)) is obtained over a well network using one or more image capturing devices (e.g., image capturing device A (141), image capturing device B (142)). Cutting image data may include an image, an image frame, a stream of image frames, and/or a portion of image, such as a specific region within a shale shaker. The cutting image data may be processed to determine cutting formation information from drilling fluid (e.g., drilling fluid C (186), drilling fluid D (187)) using machine learning (e.g., one or more of the machine-learning models G (117)) and/or one or more image processing functions (e.g., image processing functions I (119)). Moreover, an image capturing device may include one or more cameras, one or more image sensors, and various other sensors for acquiring image data of cuttings. In particular, image capturing devices may include smart network devices, waterproof devices, wired devices, and/or wireless devices disposed at different locations at a drilling rig site, such as being coupled to a shale shaker. As such, an image capturing device may include a hardware processor, a memory, one or more lenses, and/or a communication interface, such as a wired or wireless communication interface. Likewise, image capturing devices may acquire images in a visible light spectrum as well as infrared images, gamma ray images, CT scans, x-ray scans, etc. In some embodiments, an automated drilling manager collects the cutting image data over a well network. For example, the automated drilling manager may use an Internet of things (IOT) platform disposed at a well site to acquire cutting image data from various IoT devices in network communication with the automated drilling manager. The automated drilling manager may then use the cutting image data to adjust drilling operations, such as based on different ECD values. Moreover, an image capturing device may include a computer system similar to the computer system (702) described below in FIG. 7 and the accompanying description.

In some embodiments, an ECD value is determined using one or more ECD models (e.g., ECD models D (114)), drilling fluid data (e.g., drilling fluid data A (111)), drilling operation data (e.g., drilling operation data B (112)), well data (such as well schematics and wellbore geometry), and data derived from cutting image data (e.g., cutting image data C (113)), such as lithological parameters (e.g., lithological parameters F (116)) and/or cutting parameters (e.g., cutting parameters E (115)). Drilling fluid data may include values for various rheological and rheological-related parameters, such as plastic viscosity (PV) data, yield point (YP) data, fluid flow rate data, funnel viscosity data, mud weight values, etc. Drilling operation data may include rate of penetration (ROP) of a drill string, average cutting size, cutting particle sizes, etc. Drilling operation data may also include well data, such as hole inclination data, pipe diameter data, wellbore depth data, wellbore geometry data, etc. In some embodiments, an automated drilling manager may use this aggregated drilling operation data, cutting image data, and/or drilling fluid data to merge analytical operations with a drilling simulator or well control simulator for understanding how the downhole environment changes while drilling.

In some embodiments, an automated drilling manager includes functionality for using one or more hole cleaning models to determine one or more hole cleaning efficiency (HCE) values based on ECD values. For example, a hole cleaning model may describe how drilling fluids under various laminar-flow regimes remove cuttings produced from drilling. As such, a hole cleaning model may characterize hole cleaning efficiency in the eccentric annuli of extended-reach well bores, evaluate drilling fluid performance, and/or predict various fluid rheological properties for optimum cleaning. Accordingly, hole cleaning models may be used in prewell planning as well as analyzing the cleaning state of a wellbore in real-time. Thus, efficient hole cleaning may affect the quality of directing and extended-reach drilling operations.

In some embodiments, an automated drilling manager transmits one or more commands (e.g., drilling system commands X (123)) to various control systems in a well system (e.g., drilling system A (120), automated material transfer system A (135), automated mud property system B (130)) in order to produce drilling operations with specific drilling parameters and/or produce drilling fluids (e.g., drilling fluid A (181), drilling fluid B (182)) having specific drilling fluid properties. Commands may include data messages transmitted over one or more network protocols using a network interface, such as through wireless data packets. Likewise, a command may also be a control signal, such as an analog electrical signal, that triggers one or more operations in a particular control system (e.g., drilling system A (120)).

Furthermore, drilling fluid data (e.g., drilling fluid data A (111)) may correspond to different physical qualities associated with drilling mud, such as specific gravity values (also referred to as mud weight or mud density), viscosity levels, pH levels, rheological values such as flow rates, temperature values, resistivity values, mud mixture weights, mud particle sizes, and various other attributes that affect the role of drilling fluid in a wellbore. For example, a drilling fluid property may be selected by a user device to have a desired predetermined rheological value, which may include a range of acceptable values, a specific threshold value that should be exceeded, a precise scalar quantity, etc. As such, an automated drilling manager or another control system may obtain sensor data (e.g., drilling fluid sensor data A (173)) from various mud property sensors (e.g., mud property sensors A (161), mud property sensors B (162)) regarding various drilling fluid property parameters. Examples of mud property sensors include pH sensors, density sensors, rheological sensors, volume sensors, weight sensors, flow meters, such as an ES flow sensor, etc. Likewise, sensor data may refer to both raw sensor measurements and/or processed sensor data associated with one or more drilling fluid properties.

With respect to mud pump systems, a mud pump system (e.g., mud pump system X (170)) may include hardware and software with functionality for supplying drilling fluid to a wellbore at one or more predetermined pressures and/or at one or more predetermined flow rates. For example, a mud pump system may include one or more displacement pumps that inject the drilling fluid into a wellbore. Likewise, a mud pump system may include a pump controller that includes hardware and/or software for adjusting local flow rates and pump pressures, e.g., in response to a command from an automated drilling manager or other control system. For example, a mud pump system may include one or more communication interfaces and/or memory for transmitting and/or obtaining data over a well network. A mud pump system may also obtain and/or store sensor data from one or more sensors coupled to a wellbore regarding one or more pump operations. While a mud pump system may correspond to a single pump, in some embodiments, a mud pump system may correspond to multiple pumps.

With respect to mixing tanks, a mixing tank may be a container or other type of receptacle (e.g., a mud pit) for mixing various liquids, fresh mud, recycled mud, additives, and/or other chemicals to produce a particular type of drilling fluid (e.g., drilling fluid A (181), drilling fluid B (182)). For example, a mixing tank may be coupled to one or more mud supply tanks, one or more additive supply tanks, one or more dry/wet feeders, and one or more control valves for managing the mixing of chemicals within a respective mixing tank. Control valves may be used to meter chemical inputs into a mixing tank, as well as release drilling fluid into a mixing tank. Likewise, a mixing tank may include and/or be coupled to various types of drilling fluid equipment not shown in FIG. 1, such as various mud lines, liquid supply lines, and/or other mixing equipment.

In some embodiments, a well system includes an automated material transfer system (e.g., automated material transfer system A (135)). In particular, an automated material transfer system may be a control system with functionality for managing supplies of bulk powder and other inputs for producing a preliminary mud mixture. For example, an automated material transfer system may include a pneumatic, conveyer belt or a screw-type transfer system (e.g., using a screw pump) that transports material from a supply tank upon a command from a sensor-mediated response. Thus, the automated material transfer system may monitor a mixing tank using weight sensors and/or volume sensors to meter a predetermined amount of bulk powder to a selected mixing tank.

Likewise, a well system may also include an automated mud property system (e.g., automated mud property system B (130)) to control the supply of various additives to a mixing tank. In some embodiments, for example, an automated mud property system may include hardware and/or software with functionality for automatically supplying and/or mixing weighting agents, buffering agents, rheological modifiers, and/or other additives until a mud mixture matches and/or satisfies one or more desired drilling fluid properties. Examples of weighting agents may include barite, hematite, calcium carbonate, siderite, etc. A buffering agent may be a pH buffering agent that causes a mud mixture to resist changes in pH levels. For example, a buffering agent may include water, a weak acid (or weak base) and salt of the weak acid (or a salt of weak base). Rheological modifiers may include drilling fluid additives that adjust one or more flow properties of a drilling fluid. One type of rheological modifier is a viscosifier, which may be an additive with functionality for providing thermal stability, hole-cleaning, shear-thinning, improving carrying capacity as well as modifying other attributes of a drilling fluid. Examples of viscosifiers include bentonite, inorganic viscosifiers, polymeric viscosifiers, low-temperature viscosifiers, high-temperature viscosifiers, oil-fluid liquid viscosifiers, organophilic clay viscosifiers, and biopolymer viscosifiers.

Furthermore, an automated drilling manager may monitor various drilling fluid properties and drilling parameters in real-time. For example, drilling fluid properties may be monitored using one or more mud property sensors. Likewise, drilling parameters may be modified in real-time based on downhole sensors, drilling sensors (e.g., using drilling sensor data X (124)), etc. In some embodiments, for example, the automated drilling manager modifies drilling fluid properties and drilling parameters at predetermined intervals until user-defined properties are achieved by the well system (100). The user-defined properties may correspond to a selection by a user device (e.g., user selection Y (192) obtained by user device Y (190) using a graphical user interface Y (191)). For example, an automated drilling manager may be coupled to a user device e.g., over a well network, or remotely (e.g., through a remote connection using Internet access or a wireless connection at a well site). Based on real-time updates received for a current drilling operation, a user and/or the automated drilling manager may modify previously-selected drilling fluid property values and/or drilling parameters, e.g., in response to changes in drilling fluid within the wellbore.

Keeping with FIG. 1, an automated drilling manager, an automated material transfer system, and/or an automated mud property system may include one or more control systems that include one or more programmable logic controllers (PLCs). Specifically, a programmable logic controller may control valve states, fluid levels, pipe pressures, warning alarms, and/or pressure releases throughout a well system. In particular, a programmable logic controller may be a ruggedized computer system with functionality to withstand vibrations, extreme temperatures, wet conditions, and/or dusty conditions, for example, around a drilling rig. In some embodiments, the automated drilling manager (110), the automated material transfer system A (135), the automated mud property system B (130), and/or the user device Y (190) may include a computer system that is similar to the computer system (702) described below with regard to FIG. 7 and the accompanying description.

Turning to FIG. 2, FIG. 2 illustrates systems in accordance with one or more embodiments. As shown in FIG. 2, a drilling system (200) may include a top drive drill rig (210) arranged around the setup of a drill bit logging tool (220). A top drive drill rig (210) may include a top drive (211) that may be suspended in a derrick (212) by a travelling block (213). In the center of the top drive (211), a drive shaft (214) may be coupled to a top pipe of a drill string (215), for example, by threads. The top drive (211) may rotate the drive shaft (214), so that the drill string (215) and a drill bit logging tool (220) cut the rock at the bottom of a wellbore (216). A power cable (217) supplying electric power to the top drive (211) may be protected inside one or more service loops (218) coupled to a control system (244). As such, drilling fluid may be pumped into the wellbore (216) using the drive shaft (214) and/or the drill string (215). Likewise, the drilling system may also include a mud pump, a mud line, mud pits, a mud return, and other components related to the circulation or recirculation of drilling fluid within the wellbore (216). The control system (244) may be similar to various control systems described above in FIG. 1 and the accompanying description, such as the automated drilling manager (110), the automated material transfer system A (135) and/or the automated mud property system B (130).

Moreover, when completing a well, casing may be inserted into the wellbore (216). The sides of the wellbore (216) may require support, and thus the casing may be used for supporting the sides of the wellbore (216). As such, a space between the casing and the untreated sides of the wellbore (216) may be cemented to hold the casing in place. The cement may be forced through a lower end of the casing and into an annulus between the casing and a wall of the wellbore (216). More specifically, a cementing plug may be used for pushing the cement from the casing. For example, the cementing plug may be a rubber plug used to separate cement slurry from other fluids, reducing contamination and maintaining predictable slurry performance. A displacement fluid, such as water, or an appropriately weighted drilling fluid, may be pumped into the casing above the cementing plug. This displacement fluid may be pressurized fluid that serves to urge the cementing plug downward through the casing to extrude the cement from the casing outlet and back up into the annulus.

As further shown in FIG. 2, sensors (221) may be included in a sensor assembly (223), which is positioned adjacent to a drill bit (224) and coupled to the drill string (215). Sensors (221) may also be coupled to a processor assembly (223) that includes a processor, memory, and an analog-to-digital converter (222) for processing sensor measurements. For example, the sensors (221) may include acoustic sensors, such as accelerometers, measurement microphones, contact microphones, and hydrophones. Likewise, the sensors (221) may include other types of sensors, such as transmitters and receivers to measure resistivity, gamma ray detectors, etc. The sensors (221) may include hardware and/or software for generating different types of well logs (such as acoustic logs or density logs) that may provide well data about a wellbore, including porosity of wellbore sections, gas saturation, bed boundaries in a geologic formation, fractures in the wellbore or completion cement, and many other pieces of information about a formation. If such well data is acquired during drilling operations (i.e., logging-while-drilling), then the information may be used to make adjustments to drilling operations in real time. Such adjustments may include rate of penetration (ROP), drilling direction, altering mud weight, and many others drilling parameters.

In some embodiments, acoustic sensors may be installed in a drilling fluid circulation system of a drilling system (200) to record acoustic drilling signals in real-time. Drilling acoustic signals may transmit through the drilling fluid to be recorded by the acoustic sensors located in the drilling fluid circulation system. The recorded drilling acoustic signals may be processed and analyzed to determine well data, such as lithological and petrophysical properties of the rock formation. This well data may be used in various applications, such as steering a drill bit using geosteering, casing shoe positioning, etc.

The control system (244) may be coupled to the sensor assembly (223) in order to perform various program functions for up-down steering and left-right steering of the drill bit (224) through the wellbore (216). More specifically, the control system (244) may include hardware and/or software with functionality for geosteering a drill bit through a formation in a lateral well using sensor signals, such as drilling acoustic signals or resistivity measurements. For example, the formation may be a reservoir region, such as a pay zone, bed rock, or cap rock.

Turning to geosteering, geosteering may be used to position the drill bit (224) or drill string (215) relative to a boundary between different subsurface layers (e.g., overlying, underlying, and lateral layers of a pay zone) during drilling operations. In particular, measuring rock properties during drilling may provide the drilling system (200) with the ability to steer the drill bit (224) in the direction of desired hydrocarbon concentrations. As such, a geosteering system may use various sensors located inside or adjacent to the drilling string (215) to determine different rock formations within a well path. In some geosteering systems, drilling tools may use resistivity or acoustic measurements to guide the drill bit (224) during horizontal or lateral drilling.

In some embodiments, a user device (e.g., user device Y (190) may provide a graphical user interface (e.g., graphical user interface Y (191)) for communicating with an automated drilling manager, e.g., to monitor drilling operations, drilling fluid operations, and equivalent circulating density data (e.g., ECD data Y (109)). For example, a user device may be a personal computer, a human-machine interface, a smartphone, or another type of computer device for presenting information and obtaining user inputs regarding the presented information. Likewise, the user device may obtain various user selections (e.g., user selections Y (192)) regarding drilling operations, drilling fluid operations, and/or hole cleaning operations. Likewise, the user device may display various reports that may include charts as well as other arrangements of well data (e.g., drilling operation reports Y (193) includes ROP values Y (194) and ECD values Y (195)).

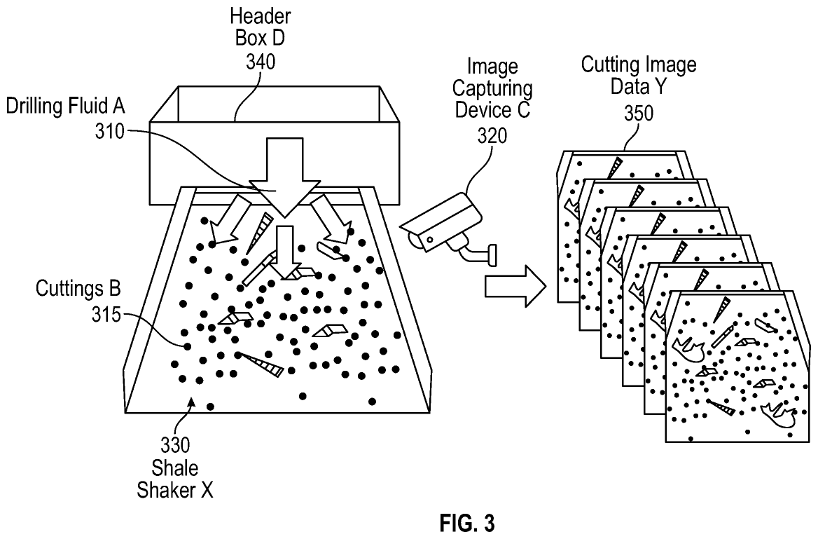

Turning to FIG. 3, FIG. 3 shows a schematic diagram in accordance with one or more embodiments. In FIG. 3, a shale shaker (e.g., shale shaker X (330)) may have a surface screen formed in a fine mesh and/or a course mesh that allows drilling fluid (e.g., drilling fluid A (310)) to pass through without various cuttings (e.g., cuttings B (315)). The shale shaker may have solids loading, solids discharge, a possum belly (e.g., a metal container at the head of the shale shaker that receives the flow of drilling fluid and is directly connected to and at the end of the flow line), and a header box (e.g., header box D (340)) to facilitate removing the cuttings from cuttings-laden drilling fluid. To clean the cuttings-laden mud, a mud circulation system may use other cleaning apparatus such as a hydrocyclone, a centrifuge, a desander, and/or a desilter. As shown in FIG. 3, drilling fluid may enter a solids control process from a header box, a possum belly, and gravity-feeds into a vibrating basket of the shale shaker. A vibrational impact of drilling fluid with the screen may cause separation between liquid and solid. The solids may be discarded while the drilling fluid and fine solids may pass into a sump tank for further treatment. Once the cuttings are disposed in the shale shaker, one or more image capturing devices (e.g., image capturing device C (320)) may acquire cutting image data (e.g., cutting image day Y (350)) for use in one or more image processing functions and/or with one or more machine-learning models to predict cutting parameters and/or lithological parameters.

Returning to FIG. 1, automated drilling manager may include hardware and/or software with functionality for generating one or more machine-learning models (e.g., machine-learning models G (117)) for use in analyzing drilling fluid and wellbore characteristics based on cutting image data (e.g., cutting image data C (113)), drilling operation data (e.g., drilling operation data B (112)), drilling fluid data (e.g., drilling fluid data A (111)). For example, automated drilling manager may use and/or process cutting image data as well as other types of data to generate and/or update one or more machine-learning models. Thus, different types of machine-learning models may be trained, such as convolutional neural networks, region-based convolutional neural networks (R-CNN), auto-encoders, deep neural networks, recurrent neural networks, support vector machines, decision trees, inductive learning models, deductive learning models, supervised learning models, unsupervised learning models, reinforcement learning models, etc. In some embodiments, two or more different types of machine-learning models are integrated into a single machine-learning architecture, e.g., a machine-learning model may include decision trees and neural networks. In some embodiments, the automated drilling manager may generate augmented or synthetic data to produce a large amount of interpreted data for training a particular model.

With respect to neural networks, for example, a neural network may include one or more hidden layers, where a hidden layer includes one or more neurons. A neuron may be a modelling node or object that is loosely patterned on a neuron of the human brain. In particular, a neuron may combine data inputs with a set of coefficients, i.e., a set of network weights and biases for adjusting the data inputs. These network weights and biases may amplify or reduce the value of a particular data input, thereby assigning an amount of significance to various data inputs for a task being modeled. Through machine learning, a neural network may determine which data inputs should receive greater priority in determining one or more specified outputs of the neural network. Likewise, these weighted data inputs may be summed such that this sum is communicated through a neuron's activation function to other hidden layers within the neural network. As such, the activation function may determine whether and to what extent an output of a neuron progresses to other neurons where the output may be weighted again for use as an input to the next hidden layer.

With respect to region-based convolutional neural networks, a region-based convolutional neural network (R-CNN) may obtain an input image or other image data at an input layer. The R-CNN may then perform a selective search process to extract various regions of interest (ROIs), where an ROI may correspond to a predetermined boundary (e.g., such as a particular rectangle) of an object in the input image. For example, an input image of cuttings in a shale shaker may include a thousand regions that are being analyzed by the R-CNN. After determining the image data for different regions, respective image data for respective regions may be sent through a neural network to determine various output features. For each region's output features, a collection of support vector machines operating as classifiers may be used to determine what type of object is contained within the respective region, such as lithology parameters or cutting parameters of a cutting portion in a particular region. Moreover, various regions that are used by an R-CNN may be referred to as ‘region proposals’ that identify smaller regions of image data that possibly include objects being searched for in the input image data. To reduce the region proposals in the R-CNN, a selective search process may be used accordingly. Examples of selective search processes include various combinatorial algorithms, such as a greedy algorithm or dynamic programming algorithm. Moreover, the convolutional neural network and the support vector machines may be trained separately based on their classifying function within the R-CNN.

Furthermore, various types of region-based convolutional neural networks are contemplated. For example, an R-CNN may be a Fast R-CNN, a Faster R-CNN, a Mask R-CNN, or a you-only-look-once (YOLO) network. While a regular R-CNN may independently determine neural network features on each region of interest, a Fast R-CNN may use a neural network only once on an entire image. At the end of the convolutional neural network, an ROIPooling process may be performed, which slices out each region from the network's output tensor, reshapes the output features, and subsequently classifies the reshaped output features, such as to determine lithology parameters or cutting parameters. As in a regular R-CNN, the Fast R-CNN may also use a selective search process to generate various region proposals. In a Faster R-CNN, the Faster R-CNN may integrate the ROI generation into the convolutional neural network itself. In a YOLO network, the YOLO network may perform similar to a fully convolutional neural network, by passing the image once through the FCNN and output a particular prediction for a grid that includes bounding boxes and class probabilities for the bounding boxes. In a Mask R-CNN, the Mask R-CNN may include object instance segmentation. Object Instance Segmentation may both detect object classes (e.g., whether a portion of cutting image data is cuttings or non-cuttings) along with determining a segmenting of a mask for each object instance (e.g., the shape of a particular cutting in the cutting image data). Likewise, some machine-learning models are contemplated that perform only semantic segmentation, such as distinguishing between cutting objects and non-cutting objects within cutting image data.

In some embodiments, various types of machine learning algorithms (e.g., machine-learning algorithms H (118)) may be used to train the model, such as a backpropagation algorithm. In a backpropagation algorithm, gradients are computed for each hidden layer of a neural network in reverse from the layer closest to the output layer proceeding to the layer closest to the input layer. As such, a gradient may be calculated using the transpose of the weights of a respective hidden layer based on an error function (also called a “loss function”). The error function may be based on various criteria, such as mean squared error function, a similarity function, etc., where the error function may be used as a feedback mechanism for tuning weights in the electronic model.

In some embodiments, a machine-learning model is trained using multiple epochs. For example, an epoch may be an iteration of a model through a portion or all of a training dataset. For example, the training data may include labeled images of cuttings that are classified with one or more predetermined lithologies. As such, a single machine-learning epoch may correspond to a specific batch of training data, where the training data is divided into multiple batches for multiple epochs. Thus, a machine-learning model may be trained iteratively using epochs until the model achieves a predetermined criterion, such as predetermined level of prediction accuracy or training over a specific number of machine-learning epochs or iterations. Thus, better training of a model may lead to better predictions by a trained model.

While FIGS. 1, 2, and 3 shows various configurations of components, other configurations may be used without departing from the scope of the disclosure. For example, various components in FIGS. 1, 2, and 3 may be combined to create a single component. As another example, the functionality performed by a single component may be performed by two or more components.

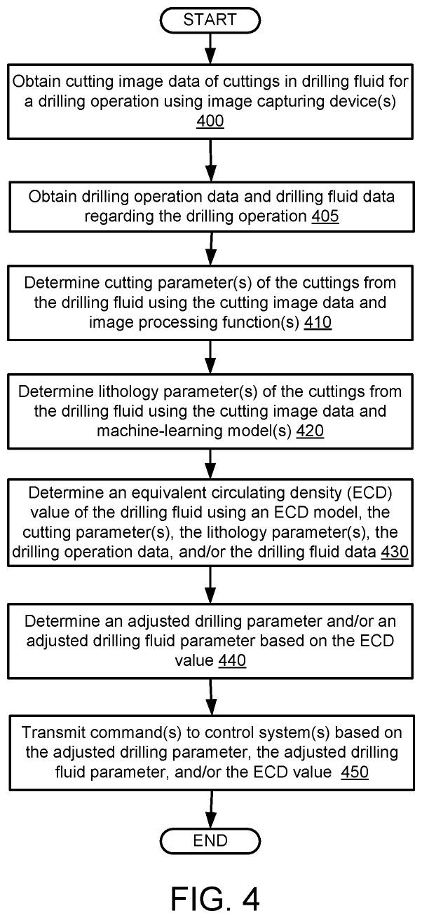

Turning to FIG. 4, FIG. 4 shows a flowchart in accordance with one or more embodiments. Specifically, FIG. 4 describes a general method for determining ECD values based on cutting-image data and/or managing a drilling operation based on the ECD values. One or more blocks in FIG. 4 may be performed by one or more components (e.g., automated drilling manager (110)) as described in FIGS. 1, 2, and 3. While the various blocks in FIG. 4 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 400, cutting image data is obtained of various cuttings in drilling fluid for a drilling operation using one or more image capturing devices in accordance with one or more embodiments. For example, the cutting image data may be acquired using one or more image capturing devices as described above in FIG. 1 and the accompanying description.

In Block 405, drilling operation data and drilling fluid data are obtained regarding a drilling operation in accordance with one or more embodiments. For example, an automated drilling manager may collect data from various sensors throughout a well site, e.g., from drilling fluid processing equipment, drilling equipment coupled to a drilling system, and downhole sensors in a wellbore. Likewise, real-time values of drilling parameters may be obtained from logging and measuring tools, surface logs, and/or daily drilling reports. Drilling operation data and/or drilling fluid data may include the rate of penetration (ROP) of a drill bit, a hole size of a wellbore, and a flow rate of the mud pump.

In Block 410, one or more cutting parameters are determined of various cuttings from drilling fluid using cutting image data and one or more image processing functions in accordance with one or more embodiments. Cutting parameters may include such as roundness, circularity, density, and volume concentration of cuttings in drilling fluid acquired from a wellbore. Likewise, image processing functions may include various image processing techniques, such as pixilation techniques. In a pixilation technique, various pixel values in a bitmap or other image data are analyzed for particular shapes, sizes, colors, etc. Based on the pixel values, the shape or size of individual cuttings may be determined. Likewise, image processing functions may also use one or more machine-learning models, such as for semantic segmentation or object instance segmentation, to determine cutting parameters in drilling fluid.

In Block 420, one or more lithology parameters are determined of various cuttings from drilling fluid using cutting image data and one or more machine-learning models in accordance with one or more embodiments. Lithology parameters may describe different lithologies and rock types found across a length of a wellbore. For example, identifying lithology parameters may reduce the uncertainty in determining the density of a particular rock formation. Likewise, lithology parameters may be used to determine ECD values by providing density, crystalline structure, friction coefficient, and other geomechanical or geochemical properties of cuttings and the related drilling fluid. As such, lithological property data may determine the density of cuttings that can alter the effective density of a drilling fluid system (e.g., affecting pressures on the formation).

Furthermore, geological formations may consist of different lithological facies composed of unique chemical compositions. While those lithological facies might be a unique composition for a given field and location, the facies may have a bounded range of specific gravity as provide in Table 1 below. Knowing the lithology in real-time may increase the accuracy of determining the specific gravity and of determining the density of the rock being drilled. Knowing the lithology parameters of cuttings may yield a more accurate real-time ECD model. The camera disposed along the mud circulation path, such as at the shale shaker, and the software for the identification of the lithology in real-time may enable the ECD model to account for the specific gravity and rock density of the cuttings. Thus, the lithology parameters may be used for modeling the behavior of the cuttings in the cuttings-laden mud in the annulus.

TABLE 1
Summary of specific gravity ranges
of four major lithology categories.

| Lithology Classification | Specific Gravity Range |
|---|---|
| Salt (Gypsum/Anhydrites) | 2.3-2.8 |
| Shale | 2.4-2.8 |
| Sandstone | 2.2-2.8 |
| Carbonates | 2.4-2.9 |

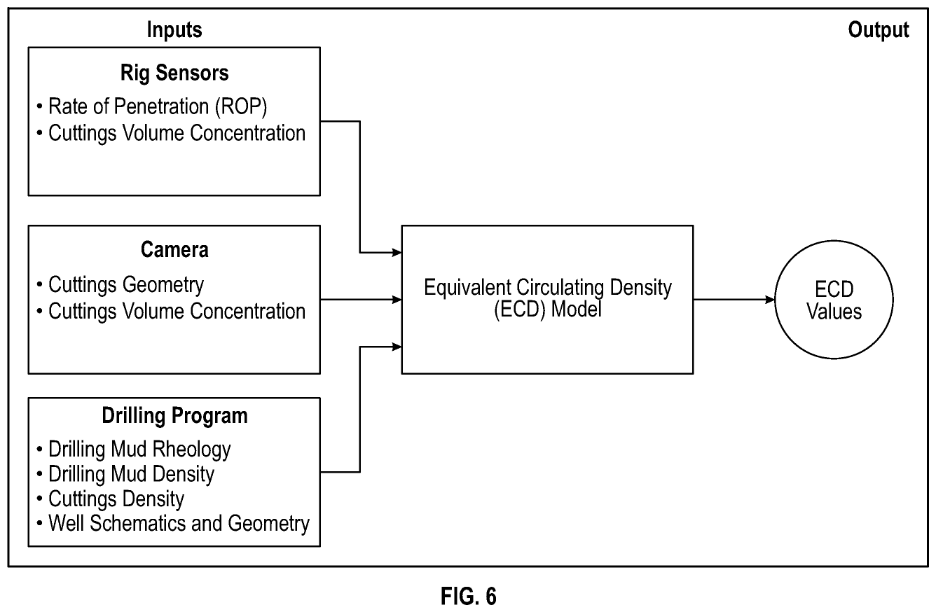

In Block 430, an equivalent circulating density (ECD) value of drilling fluid using an ECD model, one or more cutting parameters, one or more lithology parameters, drilling operation data, and/or drilling fluid data in accordance with one or more embodiments. For example, FIG. 6 shows an example of determining ECD values using rate of penetration (ROP) data and cutting volume concentration data from sensors or a drilling rig, cutting geometry data and cutting volume concentration data based on cutting image data, and data regarding a drilling program for a drilling operation. For more information on ECD models, see the section below titled “Equivalent Circulating Density (ECD) of Drilling Fluid” and the accompanying description.

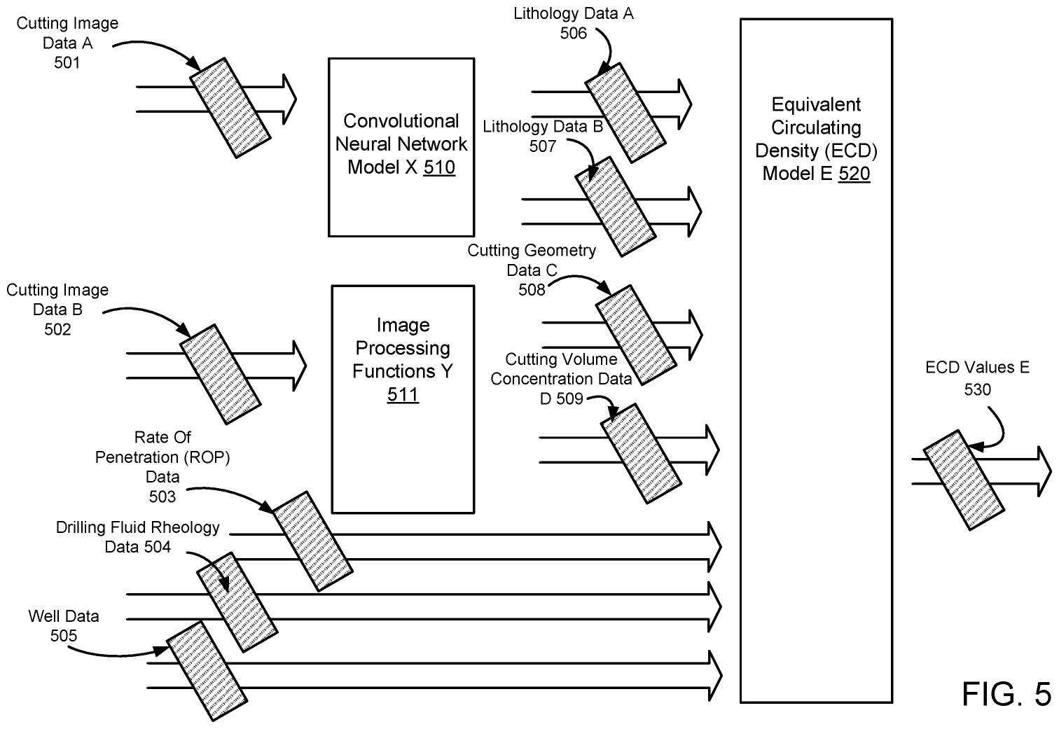

Turning to FIG. 5, FIG. 5 provides an example of determining an ECD values using cutting image data, image processing functions, and machine learning in accordance with one or more embodiments. The following example is for explanatory purposes only and not intended to limit the scope of the disclosed technology. In FIG. 5, a convolutional neural network model X (510) obtains various cutting image data A (501) from various image capturing devices (not shown) of cuttings acquired in real-time during a drilling operation. The convolutional neural network model X (510) subsequently outputs various lithological parameters based on the cuttings, i.e., lithological data A (506) and lithological data B (507). Likewise, various image processing functions Y (511) also obtain cutting image data B (502) of cuttings and determines various cutting parameters accordingly, i.e., cutting geometry data C (508) and cutting volume concentration data D (509). Using lithological data A (506), lithological data B (507), cutting geometry data C (508), cutting volume concentration data D (509), rate of penetration data (503), drilling fluid rheology data (504) and well data (505), such as well schematic data or wellbore geometry data, an ECD model E (520) determines various ECD values E (530). The ECD values E (530) may subsequently be used to adjust an ongoing drilling operation, such as to modify drilling fluid parameters in the drilling operation or the rate of penetration of the drill string.

Returning to FIG. 4, in Block 440, an adjusted drilling parameter and/or an adjusted drilling fluid parameter are determined based on an ECD value in accordance with one or more embodiments. For example, drilling parameters and drilling fluid parameters may be adjusted in real-time to improve hole cleaning efficiency based on one or more ECD values. Likewise, by detecting the current ECD state of a wellbore in real-time, retreatment operations may be automated, e.g., by adjusting various drilling fluid properties to account for changes in cutting particle sizes.

In some embodiments, a user may select within a graphical user interface different drilling parameter values or different drilling fluid parameter values to achieve different ECD values. This selection may be part of the request from a user device to adjust the current ECD value being detected in a wellbore. In another example, a user device or a control system in a well system may automatically determine an adjusted value that achieves a specified ECD value, e.g., based on a formation type or a particular well path design.

In Block 450, one or more commands are transmitted to one or more control systems based on an adjusted drilling parameter, an adjusted drilling fluid parameter, and/or an ECD value in accordance with one or more embodiments. In response to a user selection of an adjusted ROP value, adjusted drilling parameter value, an adjusted drilling fluid value, or a decision automatically made by an automated drilling manager or another control system, a command may be transmitted to one or more components within a drilling system in order to achieve the adjusted values.

### Equivalent Circulating Density (ECD) of Drilling Fluid

During a drilling operation, hydrostatic pressure may refer to the pressure exerted by the drilling fluid in static conditions, which may be expressed using the following equation:

ΔP=MW×Depth×0.052 Equation 1

where MW is mud weight of a drilling fluid (e.g., in parts per gallon (ppg)) and depth corresponds to a particular depth of a wellbore. On the other hand, equivalent circulating density (ECD) may be a dynamic density such as when the cuttings ratio and composition within the drilling fluid changes during the drilling operation. Unlike hydrostatic pressure, ECD may be the sum of the static mud weight plus the annular frictional pressure loss in dynamic conditions, and the surface back-pressure. Factors that affect annular frictional pressure loss may include annular clearance, annular velocity, mud rheology, mud weight, and measured depth of the well (MD). An ECD value may be expressed using the following equation:

$$ECD = MW + \frac{Δ {P}_{frictional}}{Depth \times 0.052} \qquad Equation 2$$

where MW corresponds to mud weight and ΔP_{frictional} corresponds to the change annular frictional pressure loss. If the ECD value rises above fracture pressure, then the rock formation may fracture and start losing drilling fluid. The ECD model may determine the ECD value at true vertical depth (TVD) and along the entire length (MD) of the well. Thus, ECD values may be monitored along the length of the well because near the drill bit area may not be the weakest point in the well. Shallower zones may be more susceptible to induced fractures due to weaker formation strengths.

While some ECD models assume various cutting parameters, embodiments disclosed herein may describe one or more ECD models that measure the geometry, size, and lithology of cuttings in real-time. Cutting parameters, such as geometrical characteristics of individual cuttings and cutting concentrations, may be used to enhance the accuracy of the ECD models. In addition, determining the geometry and shape of cuttings may result in a better prediction of a shape friction factor. Different shapes such as angular, blocky, rounded, splintery, and tabular may have different friction factor values that may lead to different annular pressure loss values. Angular may refer to cuttings with a triangular or arrowhead shape and a rough surface structure. Blocky cuttings may be cubic in structure. Splintery cuttings may have flat, thin, and planar structures. Tabular cuttings may have flat, parallel faces. Rounded cuttings may have round corners and edges.

Cutting concentration measurements may be physics-based calculations and may be a function of rate of penetration and drilling fluid flowrate. The camera-based cuttings analysis tool may enable a human operator or automated system to determine in real-time and in dynamic conditions the actual cuttings volume concentration (vs. estimated cuttings volume concentration, a drilling parameter) at points along the mud circulation path such as returning to the shale shaker. Quantifying the cuttings volume percentage in dynamic conditions may improve the accuracy of the ECD model.

One direct usage of the image-based cuttings analysis tool may be to measure the cuttings volume concentration in the well to obtain a measured cuttings volume concentration. For example, the yield stress for fluids with a suspended material may be a function of new substances and additives that alter the rheology of a fluid. The yield stress may be expressed using the following equation:

$${τ}_{y} ( α ) {τ}_{0} \sqrt{( 1 - α ) {( 1 - \frac{α}{β} )}^{- 2.5 β}} \qquad Equation 3$$

where τ_{y} corresponds to an effective yield stress, τₒ corresponds to a measured yield stress value, α is the volume fraction of solids (e.g., as measured by an image-based cuttings analysis tool), and β is an experimental constant that may be equal to 0.905. A consistency index K may also be a function of solid volume fraction, α, which may be expressed using the following equation

$$K ( α ) = {K}_{o} \sqrt{{( 1 - α )}^{1 - m} {( 1 - \frac{α}{β} )}^{- 2.5 β ( m + 1 )}} \qquad Equation 4$$

where K(α) corresponds to an effective consistency constant of a drilling fluid and Kₒ corresponds to a measured consistency constant.

Furthermore, cuttings slip velocity may refer to the rate at which cuttings slip behind a liquid state, which may also be known as slip rate. One primary function of drilling fluids is the removal of cuttings from bottom-hole to surface. Cuttings tend to fall downward in vertical wells due to gravity and buoyancy effects. The movement of cuttings in the well may depend on the slip velocity and the upward movement of drilling fluid. If the drilling fluid velocity is greater than cuttings settling velocity, then the resultant cuttings velocity may have upward movement where cuttings may eventually reach surface. However, if the cuttings settling velocity is greater than drilling fluid velocity, for example, in cases of low fluid velocities or cases of no fluid circulation, then cuttings may fall down toward bottom-hole.

Cuttings slip velocity may be a function of several inputs, one of which is the cuttings size. For example, cuttings slip velocity may be determined by applying a force balance on a spherical particle in Newtonian fluids, where the Reynolds Particle number for Newtonian fluids may be expressed as the following equation:

$${Re}_{p} = \frac{928 {ρ}_{f} {v}_{sl} {d}_{s}}{μ} \qquad Equation 5$$

where dₛ is the particle diameter of cuttings, μ is the Newtonian fluid viscosity, νₛₜ is the particle settling velocity, where ρ_{f} is the density of the fluid, and Reₚ corresponds to the Reynolds Particle number. The particle settling velocity may be expressed using the following equation:

$${v}_{sl} = \frac{138 ( {ρ}_{s} - {ρ}_{f} ) {d}_{s}^{2}}{μ} \qquad Equation 6$$

where ρₛ is the density of a cutting particle. Moreover, a friction factor can be determined using the following equation:

$$f = 3.57 \frac{{d}_{s}}{{v}_{sl}^{2}} \frac{{ρ}_{s} - {ρ}_{f}}{{ρ}_{f}} \qquad Equation 7$$

where ρ_{f} is the density of a fluid. Likewise, the Newtonian fluid viscosity may be expressed using the following equation:

$${v}_{sl} = 1.89 \sqrt{\frac{{d}_{s}}{f} \frac{{ρ}_{s} - {ρ}_{f}}{{ρ}_{s}}} \qquad Equation 8$$

Many drilling fluids may be shear-thinning fluids. Thus, a viscosity of a shear-thinning fluid may decrease under shear strain, i.e., with a higher shear rate, thus the apparent viscosity varies with different shear rate values. The Yield Power Law (YPL) model, also known as the Herschel-Bulkley (H-B) model, may be expressed using the following equation:

τ=τ_{y}+K\dot{γ}^{m} Equation 9

where the apparent viscosity η may be expressed using the following equation:

$$η = \frac{τ}{\dot{γ}} \qquad Equation 10$$

where \dot{γ} corresponds to a shear rate. Using the YPL model, an apparent viscosity η may be expressed using the following equation:

$$η = \frac{τ}{\dot{γ}} = \frac{{τ}_{y}}{\dot{γ}} + K {\dot{γ}}^{m - 1} \qquad Equation 11$$

Moreover, YPL fluids flowing in an annulus may be expressed using the following equation:

$$\dot{γ} = ( \frac{2 N + 1}{3 N} ) \frac{12 V}{D} \qquad Equation 12$$

where N corresponds to generalized flow behavior index, V corresponds to the drilling mud annular velocity, and D corresponds to the annulus hydraulic diameter. The apparent viscosity of shear thinning fluids may be used in Bourgoyne's Newtonian fluids slip velocity to determine the slip velocities for Yield Power Law fluids.

During a drilling operation, an automated drilling manager or other control system may determine, in real time, the ECD of a drilling fluid. In an example, based on an ECD value, the drilling system may determine a maximum rate of penetration for a drill bit. More specifically, the ECD, a pore pressure limit of the formation, and a fracture pressure limit of the formation are used to calculate the stability of the formation. Then, based on the calculated stability, the maximum rate of penetration may be calculated. Additionally, the drilling system may control the rate of penetration, perhaps to be less than the calculated maximum rate. Controlling the rate of penetration based on ECD values may allow a drilling system to: (i) avoid fracturing the formation while drilling, (ii) ensure smooth drilling with generated drilling cuttings, and (iii) avoid or mitigate stuck pipe incidents.

In another example, based on the current value of the ECD, the drilling system may adjust drilling parameters and/or drilling fluid parameters to produce a different ECD value. In one implementation, the drilling system adjusts the ECD by controlling a mud pump to increase or decrease the volume of drilling fluid pumped into the wellbore, thereby increasing or decreasing the effective drilling fluid density. Increasing the volume of drilling fluid decreases the drilling fluid density by dilution and decreasing the volume of drilling fluid increases the drilling fluid density. In another implementation, the drilling system adjusts an ECD value by increasing the drilling fluid density by adding a weighing agent to the drilling fluid. In yet another implementation, the drilling system adjusts the ECD by controlling one of the drilling pipe outer diameter, the yield point of the drilling fluid, the plastic viscosity of the drilling fluid, or the annular velocity of the drilling fluid.

### Computer System

Embodiments may be implemented on a computer system. FIG. 7 is a block diagram of a computer system (702) used to provide computational functionalities associated with described algorithms, methods, functions, processes, flows, and procedures as described in the instant disclosure, according to an implementation. The illustrated computer system (702) is intended to encompass any computing device such as a high-performance computing (HPC) device, a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer system (702) may include a computer that includes an input device, such as a keypad, keyboard, touch screen, or other device that can accept user information, and an output device that conveys information associated with the operation of the computer system (702), including digital data, visual, or audio information (or a combination of information), or a GUI.

The computer system (702) can serve in a role as a client, network component, a server, a database or other persistency, or any other component (or a combination of roles) of a computer system for performing the subject matter described in the instant disclosure. The illustrated computer system (702) is communicably coupled with a network (730) or cloud. In some implementations, one or more components of the computer system (702) may be configured to operate within environments, including cloud-computing-based, local, global, or other environment (or a combination of environments).

At a high level, the computer system (702) is an electronic computing device operable to receive, transmit, process, store, or manage data and information associated with the described subject matter. According to some implementations, the computer system (702) may also include or be communicably coupled with an application server, e-mail server, web server, caching server, streaming data server, business intelligence (BI) server, or other server (or a combination of servers).

The computer system (702) can receive requests over network (730) or cloud from a client application (for example, executing on another computer system (702)) and responding to the received requests by processing the said requests in an appropriate software application. In addition, requests may also be sent to the computer system (702) from internal users (for example, from a command console or by other appropriate access method), external or third-parties, other automated applications, as well as any other appropriate entities, individuals, systems, or computers.

Each of the components of the computer system (702) can communicate using a system bus (703). In some implementations, any or all of the components of the computer system (702), both hardware or software (or a combination of hardware and software), may interface with each other or the interface (704) (or a combination of both) over the system bus (703) using an application programming interface (API) (712) or a service layer (713) (or a combination of the API (712) and service layer (713). The API (712) may include specifications for routines, data structures, and object classes. The API (712) may be either computer-language independent or dependent and refer to a complete interface, a single function, or even a set of APIs. The service layer (713) provides software services to the computer system (702) or other components (whether or not illustrated) that are communicably coupled to the computer system (702). The functionality of the computer system (702) may be accessible for all service consumers using this service layer. Software services, such as those provided by the service layer (713), provide reusable, defined business functionalities through a defined interface. For example, the interface may be software written in JAVA, C++, or other suitable language providing data in extensible markup language (XML) format or other suitable format. While illustrated as an integrated component of the computer system (702), alternative implementations may illustrate the API (712) or the service layer (713) as stand-alone components in relation to other components of the computer system (702) or other components (whether or not illustrated) that are communicably coupled to the computer system (702). Moreover, any or all parts of the API (712) or the service layer (713) may be implemented as child or sub-modules of another software module, enterprise application, or hardware module without departing from the scope of this disclosure.

The computer system (702) includes an interface (704). Although illustrated as a single interface (704) in FIG. 7, two or more interfaces (704) may be used according to particular needs, desires, or particular implementations of the computer system (702). The interface (704) is used by the computer system (702) for communicating with other systems in a distributed environment that are connected to the network (730). Generally, the interface (704 includes logic encoded in software or hardware (or a combination of software and hardware) and operable to communicate with the network (730) or cloud. More specifically, the interface (704) may include software supporting one or more communication protocols associated with communications such that the network (730) or interface's hardware is operable to communicate physical signals within and outside of the illustrated computer system (702).

The computer system (702) includes at least one computer processor (705). Although illustrated as a single computer processor (705) in FIG. 7, two or more processors may be used according to particular needs, desires, or particular implementations of the computer system (702). Generally, the computer processor (705) executes instructions and manipulates data to perform the operations of the computer system (702) and any algorithms, methods, functions, processes, flows, and procedures as described in the instant disclosure.

The computer system (702) also includes a memory (706) that holds data for the computer system (702) or other components (or a combination of both) that can be connected to the network (730). For example, memory (706) can be a database storing data consistent with this disclosure. Although illustrated as a single memory (706) in FIG. 7, two or more memories may be used according to particular needs, desires, or particular implementations of the computer system (702) and the described functionality. While memory (706) is illustrated as an integral component of the computer system (702), in alternative implementations, memory (706) can be external to the computer system (702).

The application (707) is an algorithmic software engine providing functionality according to particular needs, desires, or particular implementations of the computer system (702), particularly with respect to functionality described in this disclosure. For example, application (707) can serve as one or more components, modules, applications, etc. Further, although illustrated as a single application (707), the application (707) may be implemented as multiple applications (707) on the computer system (702). In addition, although illustrated as integral to the computer system (702), in alternative implementations, the application (707) can be external to the computer system (702).

There may be any number of computers (702) associated with, or external to, a computer system containing computer system (702), each computer system (702) communicating over network (730). Further, the term “client,” “user,” and other appropriate terminology may be used interchangeably as appropriate without departing from the scope of this disclosure. Moreover, this disclosure contemplates that many users may use one computer system (702), or that one user may use multiple computers (702).

In some embodiments, the computer system (702) is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, a cloud computing system may operate according to one or more service models, such as infrastructure as a service (IaaS), platform as a service (PaaS), software as a service (Saas), mobile backend as a service (MBaaS), artificial intelligence as a service (AIaaS), serverless computing, and/or function as a service (FaaS).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method, comprising:
obtaining, using an image capturing device, first cutting image data regarding a plurality of cuttings carried by a drilling fluid circulating in a wellbore during a drilling operation;
obtaining drilling operation data in real-time regarding a drilling system performing the drilling operation;
obtaining drilling fluid data regarding the drilling fluid;
determining, by a computer processor, a first cutting parameter of the plurality of cuttings using the first cutting image data and an image processing function;
determining, by the computer processor, a lithology parameter of the plurality of cuttings using the first cutting image data and a machine-learning model;
determining, by the computer processor, an equivalent circulating density (ECD) value of the drilling fluid using an ECD model, the first cutting parameter, the lithology parameter, the drilling operation data, and the drilling fluid data; and
transmitting, using the computer processor, a command that adjusts a drilling parameter of the drilling operation based on the ECD value of the drilling fluid.

2. The method of claim 1, wherein the image capturing device is a camera device coupled to a shale shaker.

3. The method of claim 1, wherein the first cutting parameter describes a cuttings geometry of a portion of the plurality of cuttings.

4. The method of claim 1, wherein the first cutting parameter corresponds to a volume concentration of the plurality of cuttings.

5. The method of claim 1, wherein the machine-learning model is a region-based convolutional neural network (R-CNN).

6. The method of claim 1, further comprising:
obtaining training data comprising a plurality of labeled images of cuttings, wherein a respective labeled image among the plurality of labeled images corresponds to a predetermined lithology; and
performing a training operation of the machine-learning model using the training data.

7. The method of claim 1, wherein the ECD value is determined by an edge server disposed at a well site comprising the wellbore.

8. The method of claim 1, further comprising:
determining a second cutting image data regarding a second plurality of cuttings of the drilling fluid;
determining cutting geometry data of the second plurality of cuttings based on the second cutting image data; and
determining a slip rate of the second plurality of cuttings using the cutting geometry data,
wherein the ECD value is based on the slip rate of the second plurality of cuttings.

9. The method of claim 1, wherein the drilling operation data comprises a rate of penetration of a drill string in the drilling operation.

10. The method of claim 1, wherein the drilling fluid data comprises rheological data regarding the drilling fluid.

11. A system, comprising:
an image capturing device disposed on a drilling fluid circulation path of a wellbore during a drilling operation;
a drilling system comprising a drill string and a plurality of sensors, wherein the drilling system is coupled to the wellbore;
a mud pump system coupled to the wellbore, wherein the mud pump system is configured to supply drilling fluid to the wellbore; and
a server coupled to the image capturing device, the drilling system, and the mud pump system, wherein the server comprises a computer processor, the server being configured to:
obtain first cutting image data regarding a plurality of cuttings carried by a drilling fluid circulating in a well using the image capturing device;
obtain drilling operation data in real-time regarding a drilling system performing the drilling operation;
obtain drilling fluid data regarding the drilling fluid;
determine a first cutting parameter of the plurality of cuttings using the first cutting image data and an image processing function;
determine a lithology parameter of the plurality of cuttings using the first cutting image data and a machine-learning model; and
determining an equivalent circulating density (ECD) value of the drilling fluid using an ECD model, the first cutting parameter, the lithology parameter, the drilling operation data, and the drilling fluid data.

12. The system of claim 11, further comprising:
a user device coupled to the server,
wherein the user device is configured to provide a graphical user interface for presenting the ECD value to a user and obtain one or more user selections regarding an adjusted rate of penetration value in response to presenting the ECD value.

13. The system of claim 11, further comprising:
a shale shaker coupled to the image capturing device,
wherein the image capturing device comprises one or more camera devices.

14. The system of claim 11, wherein the first cutting parameter describes a cuttings geometry of a portion of the plurality of cuttings.

15. The system of claim 11, wherein the first cutting parameter corresponds to a volume concentration of the plurality of cuttings.

16. The system of claim 11, wherein the machine-learning model is a region-based convolutional neural network (R-CNN).

17. The system of claim 11, further comprising:
a control system coupled to the server,
wherein the server is configured to adjust a drilling parameter or drilling fluid parameter based on the ECD value.

18. The system of claim 11, wherein the server is configured to:
obtain training data comprising a plurality of labeled images of cuttings, wherein a respective labeled image among the plurality of labeled images corresponds to a predetermined lithology; and
perform a training operation of the machine-learning model using the training data.

19. The system of claim 11, wherein the server is configured to:
determine a second cutting image data regarding a second plurality of cuttings of the drilling fluid;
determine cutting geometry data of the second plurality of cuttings based on the second cutting image data; and
determine a slip rate of the second plurality of cuttings using the cutting geometry data,
wherein the ECD value is based on the slip rate of the second plurality of cuttings.

20. The system of claim 11, wherein the drilling operation data comprises a rate of penetration of a drill string in the drilling operation.
